(19) United States
(12) Patent Application Publication
(10) Pub. No.: US 2016/0082116 A1
(43) Pub. Date: Mar. 24, 2016

(54) ENGINEERED MICROGELS

(71) Applicants: BOARD OF REGENTS, THE UNIVERSITY OF TEXAS SYSTEM, Austin, TX (US); MICROMULSION, INC., Austin, TX (US)

(72) Inventors: Laura Suggs, Austin, TX (US); Suraj Makhija, Austin, TX (US); Nishant Mehta, Austin, TX (US); Daniel Min, Austin, TX (US); Anirudh Sharma, Flower Mound, TX (US)

(21) Appl. No.: 14/859,731

(22) Filed: Sep. 21, 2015

Related U.S. Application Data
(60) Provisional application No. 62/052,934, filed on Sep. 19, 2014.

(51) Int. Cl. A61K 47/48; A61K 47/20; C12N 5/00; A61K 31/727

(52) U.S. Cl. CPC A61K 47/48215; A61K 31/727; A61K 47/20; C12N 5/0075; C12N 2531/00; C12N 2501/91

(57) ABSTRACT

Microparticles containing heparin or a heparin-like polymer and a biocompatible polymer are described. The heparin or the heparin-like polymer and the biocompatible polymer can be indirectly linked together by a coupling agent, which can have a structure represented by Formula I, (A)ₚ(R)(D)_{q}, wherein A is a bond or a moiety that can form a bond with the heparin or the heparin-like polymer, D is a bond or a moiety that can form a bond with the biocompatible polymer, R is a linker for A and D, and p and q are from 1 to 25. Methods of making the microparticles include mixing a first solution of the heparin or the heparin-like polymer and a second solution of the biocompatible polymer, to form a mixture, and adding the mixture to an oil and a surfactant and homogenizing the mixture to form a water-in-oil emulsion. Compositions of these microparticles are also described.

### FIELD

The present disclosure relates to microparticles, particularly to microgels, and to methods of making and using such microparticles.

### BACKGROUND

A growing number of proteins and peptides have been produced by recombinant DNA technology for use as pharmaceutical therapeutic agents. Such proteins include erythropoietin (EPO), granulocyte-colony-stimulating factor (G-CSF and GM-CSF), interferons (alpha, beta, gamma, and consensus), insulin and interleukin-1 etc. In addition to these proteins, several hundred other proteins are currently undergoing clinical trials as drugs. Tissue engineering is another area of biomedical research that is much pursued. In addition to cells and their scaffolds, protein growth factors are frequently required for tissue engineering. These include nerve growth factor (NGF), platelet-derived growth factor (PDGF), fibroblast growth factor (FGF), epidermal growth factor (EGF), tumor necrosis factor (TNF) etc.

Because proteins have generally short in vivo half-lives and negligible oral bioavailability, they are typically administered by frequent injection, a procedure that is hard to accept for most patients. Similarly, cell growth factors are sensitive and easily degraded, thus, they are usually injected locally. However, it has been found that direct single-time injection of a growth factor solution into a regeneration site is less effective, as the injected growth factor rapidly diffuses away from the site. Repeated injection is, of course, inconvenient.

There remains a need in the medical arts for vehicles that are capable of releasing biomolecules such as proteins, enzymes, hormones, and peptides at a controlled and predictable rate in order-to provide effective release of the factor over a clinically useful period. There also is a need for vehicles that are capable of increasing the stability of biomolecules and simultaneously provide the release of the biomolecules at a controlled rate. The microparticles, compositions, and methods disclosed herein address these and other needs.

### SUMMARY

Microparticles and compositions comprising the microparticles are disclosed herein. The microparticles can comprise heparin or a heparin-like polymer and a biocompatible polymer. The heparin or the heparin-like polymer and the biocompatible polymer can be directly or indirectly linked. The microparticle can have an average diameter of from about 2 μm to about 30 μm.

The heparin in the described microparticles can have a molecular weight of from about 1,000 to about 50,000 Daltons, for example, from about 1,000 to about 20,000 Daltons. Similarly, the heparin-like polymer can have a molecular weight of from about 1,000 to about 50,000 Daltons, for example, from about 1,000 to about 20,000 Daltons. The heparin-like polymer can be a polysaccharide having at least one negative charge per two saccharide rings and no more than one positive charge per ten saccharide rings. For example, the heparin-like polymer can be dextran sulfates, chondroitin sulfates, heparin sulfates, fucans, and alginates. The heparin or the heparin-like polymer can be natural or synthetic.

The biocompatible polymer can have a molecular weight of from about 1,000 to about 50,000 Daltons, for example, from about 1,000 to about 30,000 Daltons. The biocompatible polymer can include a polyalkylene oxide, polylactic acid, polyacrylic acid, polyurethane, polyphosphazene, polysaccharide, dextran, polyvinyl pyrrolidone, polyvinyl alcohol, polyacrylamide, copolymers thereof, and blends thereof.

In some examples, the heparin or the heparin-like polymer and the biocompatible polymer can be linked via a coupling agent. The method for covalently attaching the heparin or the heparin-like polymer to the biocompatible polymer can be via a coupling agent. The coupling agent can have a structure represented by Formula I:

(A)ₚ(R)(D)_{q} Formula I

wherein A is a bond or a moiety that can form a bond with the heparin or the heparin-like polymer, D is a bond or a moiety that can form a bond with the biocompatible polymer, R is a linker for A and D, and p and q are integers from 1 to 25. A and D, for each occurrence, can independently include a Michael acceptor, a Michael donor, an amine containing group, a hydroxyl containing group, a thiol containing group, a carboxylic acid containing group, or combinations thereof. It is to be understood that when the coupling agent is bonded to the heparin or the heparin-like polymer and the biocompatible polymer, thus linking these two polymers together, A and D represent a moiety bonded to the heparin-like polymer and the biocompatible polymer, respectively. For example, where A is described as an amine containing group in the coupling agent, when the coupling agent is bonded to the heparin or heparin-like polymer, A is then defined as the result of a reaction between the amine containing group and the heparin or heparin-like polymer (e.g., a secondary or tertiary amine or an amide). Similarly, when D is described as a Michael acceptor in the coupling agent, when the coupling agent is bonded to the biocompatible polymer, D is then defined as the result of a reaction between the Michael acceptor and a Michael donor (e.g., an alkyl, thio ether, ether, or amine beta to a carbonyl). The linker moiety, R, can include an atom such as oxygen, sulfur, carbon, boron, or nitrogen. The linker can also include a radical such as a substituted or unsubstituted alkoxy, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heterocycloalkyl, substituted or unsubstituted heteroaryl, substituted or unsubstituted ether, or substituted or unsubstituted amine In some examples, the linker can be a polymer.

Methods of making the described microparticles are also disclosed. The method can include providing a first solution of heparin or a heparin-like polymer and a second solution of the biocompatible polymer. The first solution and the second solution can be mixed to form a mixture. An oil-in-water emulsion of the mixture can be formed by adding the mixture to an oil and/or a surfactant. Suitable oil can include paraffin oil, squalane, pristane, polyisobutene oil, hydrogenated polyisobutene oil, polydecene oil, polyisoprene oil, polyisopropene oil, and combinations thereof. Suitable surfactants can include Span, Tween and Brij surfactants. The method can also include homogenizing the oil-in-water emulsion. Homogenizing can be carried out from about 2,000 rpm to about 5,000 rpm for 10 minutes or less.

Methods of using the described microparticles are also disclosed. The microparticles can be mixed with, or be electrostatically or covalently coupled to any of a variety of therapeutic, prophylactic, or diagnostic agents or other protein bio-active agents, thus making them suitable for controlled release. The microparticles can also be used to encapsulate cells and tissues. Compounds with a wide range of molecular weight can be coupled to the microparticles, for example, from about 100 to about 500,000 grams per mole. In some examples, the microparticles can be used in cell culture media and other biological assays. For example, the microparticles disclosed herein can be used for release of cell growth factors in tissue engineering or cell cultures.

In some examples, the described microparticles can be used in medical devices for use in biodegradable implants. Devices prepared from the microparticles can include particles, matrices and/or scaffolds for delivery of a therapeutic or diagnostic agent, particularly for controlled release of an active agent, particles, matrices and/or scaffolds for tissue engineering, cell encapsulation, targeted delivery, guided tissue regeneration; and diagnostics.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a scheme showing heparin-maleimide synthesis. Heparin is added to maleimide acetate in the presence of an acid. The heparin maleimide is dialyzed against deionized water and 1M sodium chloride.

FIG. 2 is a scheme showing microgel synthesis via Michael addition reaction. In step (1), the dialyzed heparin-maleimide obtained from the scheme shown in FIG. 1 is combined with PEG-thiol in buffer to give a thioether. In step (2), the thioether is combined into a surfactant/oil solution to form a water-in-oil emulsion. In step (3), the emulsion is washed with paraffin oil and water and imaged via light microscopy.

FIG. 3A through 3C are UV/VIS spectra of enoxaparin (FIG. 3A), maleimide acetate (AEM) (FIG. 3B), and heparin maleimide (FIG. 3C). Maleimide acetate displays a distinct shoulder at approximately 300 nm as shown in FIG. 3B and FIG. 3C.

FIG. 4A and FIG. 4B are NMR spectra of heparin-maleimide obtained by Baldwin et al. (FIG. 4A) and from the scheme in FIG. 2 (FIG. 4B). The maleimide peak at approximately 6.8 ppm is present in both spectra.

FIG. 5A and FIG. 5B are UV/VIS spectra of varying concentrations of heparin (FIG. 5A) and heparin maleimide (FIG. 5B) in methylene blue. Methylene blue interacts and changes the absorption spectrum of heparin. The keys show dilution of heparin and heparin maleimide stock solutions.

FIG. 6A and FIG. 6B are images of microgels using light microscopy. Images were generated with (FIG. 6A) phase contrast and (FIG. 6B) bright field. FIG. 6C is a UV/VIS spectrum of varying concentrations of heparin maleimide microgel in methylene blue. The ratios in the key shows dilution of microgel stock solutions. The shoulder at about 550 nm is indicative of heparin.

FIG. 7A is a measurement of microgel diameter in ImageJ using the hemocytometer grid for scale. FIG. 7B is a bar graph showing the size distribution of microgel particles synthesized in TEA buffer, pH 7.8. The diameters were obtained by analyzing of images of microgels obtained from a hemocytometer using ImageJ. The diameter was 14.2±6.5 μm.

FIG. 8 is a bar graph showing the size distribution of microgel particles synthesized in TEA buffer, pH 7.5. The diameters were obtained by analyzing of images of microgels obtained from a hemocytometer using ImageJ. The diameter was 11.9±6.5 μm. the diameters of the microgel particles are smaller at pHs lower than 7.8.

FIG. 9 is a UV/VIS spectrum showing the binding profile of thrombin and heparin-maleimide microgels of varying concentrations of heparin maleimide microgel in methylene blue. The ratios in the key show the high peak to low peak ratios (low: 550-570 nm, high: 660-680 nm). The data was analyzed quantitatively by taking the numerical integration under the two peaks and then computing the high/low integral ratio.

FIG. 10 is a graph showing the protein (thrombin) quantity vs. mass microgels in samples with regression lines and equations. The slope of the regression line forced through (x,y)=(0,0) is a measure of the capacity of protein binding. Grey lines represent the 95% confidence interval for the regression line.

FIG. 11 is a graph showing the cumulative release of protein from microgels over time in the presence of 10 mM glutathione.

FIG. 12 is a graph of viability of cells exposed to microgel/medium suspensions over three days.

FIG. 13 is a histogram of microgel diameters.

### DETAILED DESCRIPTION

The microparticles, compositions, and methods described herein may be understood more readily by reference to the following detailed description of specific aspects of the disclosed subject matter and the Examples and Figures included therein.

Before the present microparticles, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the microparticle” includes mixtures of two or more such microparticles, reference to “an agent” includes mixture of two or more such agents, and the like.

“Optional” or “optionally” means that the subsequently described event or circumstance can or cannot occur, and that the description includes instances where the event or circumstance occurs and instances where it does not.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Chemical Definitions

Terms used herein will have their customary meaning in the art unless specified otherwise. The organic moieties mentioned when defining variable positions within the general formulae described herein (e.g., the term “halogen”) are collective terms for the individual substituents encompassed by the organic moiety.

The term “alkyl,” as used herein, refers to saturated straight, branched, primary, secondary or tertiary hydrocarbons, including those having 1 to 20 atoms. In some examples, alkyl groups will include C₁-C₁₂, C₁-C₁₀, C₁-C₈, C₁-C₆, C₁-C₅, C₁-C₄, C₁-C₃, or C₁-C₂ alkyl groups. Examples of C₁-C₁₀ alkyl groups include, but are not limited to, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl groups, as well as their isomers. Examples of C₁-C₄-alkyl groups include, for example, methyl, ethyl, propyl, 1-methylethyl, butyl, 1-methylpropyl, 2-methylpropyl, and 1,1-dimethylethyl groups.

Cyclic alkyl groups or “cycloalkyl” groups include cycloalkyl groups having from 3 to 10 carbon atoms. Cycloalkyl groups can include a single ring, or multiple condensed rings. In some examples, cycloalkyl groups include C₃-C₄, C₄-C₇, C₅-C₇, C₄-C₆, or C₅-C₆ cyclic alkyl groups. Non-limiting examples of cycloalkyl groups include adamantyl, cyclopropyl, cyclobutyl, cyclopentyl, cyclohexyl, cycloheptyl, cyclooctyl and the like.

Alkyl and cycloalkyl groups can be unsubstituted or substituted with one or more moieties chosen from alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the disclosure, either unprotected, or protected as necessary, as known to those skilled in the art, for example, as described in Greene, et al., Protective Groups in Organic Synthesis, John Wiley and Sons, Third Edition, 1999, hereby incorporated by reference.

Terms including the term “alkyl,” such as “alkylamino” or “dialkylamino,” will be understood to comprise an alkyl group as defined above linked to another functional group, where the group is linked to the compound through the last group listed, as understood by those of skill in the art.

The term “alkenyl,” as used herein, refers to both straight and branched carbon chains which have at least one carbon-carbon double bond. In some examples, alkenyl groups can include C₂-C₂₀ alkenyl groups. In other examples, alkenyl can include C₂-C₁₂, C₂-C₁₀, C₂-C₈, C₂-C₆, or C₂-C₄ alkenyl groups. In one example of alkenyl, the number of double bonds is 1-3, in another example of alkenyl, the number of double bonds is one or two. Other ranges of carbon-carbon double bonds and carbon numbers are also contemplated depending on the location of the alkenyl moiety on the molecule. “C₂-C₁₀-alkenyl” groups can include more than one double bond in the chain. The one or more unsaturations within the alkenyl group can be located at any position(s) within the carbon chain as valence permits. In some examples, when the alkenyl group is covalently bound to one or more additional moieties, the carbon atom(s) in the alkenyl group that are covalently bound to the one or more additional moieties are not part of a carbon-carbon double bond within the alkenyl group. Examples of alkenyl groups include, but are not limited to, ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl groups.

The term “alkynyl,” as used herein, refers to both straight and branched carbon chains which have at least one carbon-carbon triple bond. In one example of alkynyl, the number of triple bonds is 1-3; in another example of alkynyl, the number of triple bonds is one or two. In some examples, alkynyl groups include from C₂-C₂₀ alkynyl groups. In other examples, alkynyl groups can include C₂-C₁₂, C₂-C₁₀, C₂-C₈, C₂-C₆ or C₂-C₄ alkynyl groups. Other ranges of carbon-carbon triple bonds and carbon numbers are also contemplated depending on the location of the alkenyl moiety on the molecule. For example, the term “C₂-C₁₀-alkynyl” as used herein refers to a straight-chain or branched unsaturated hydrocarbon group having 2 to 10 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3 -yn-2-yl, 3 -methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl, and 4-methylpent-2-yn-5-yl groups.

The term “aryl,” as used herein, refers to a monovalent aromatic carbocyclic group of from 6 to 14 carbon atoms. Aryl groups can include a single ring or multiple condensed rings. In some examples, aryl groups include C₆-C₁₀ aryl groups. Aryl groups include, but are not limited to, phenyl, biphenyl, naphthyl, tetrahydronaphtyl, phenylcyclopropyl and indanyl. Aryl groups can be unsubstituted or substituted by one or more moieties chosen from halo, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, cycloalkoxy, cycloalkenyloxy, halocycloalkoxy, halocycloalkenyloxy, alkylthio, haloalkylthio, cycloalkylthio, halocycloalkylthio, alkylsulfinyl, alkenylsulfinyl, alkynyl-sulfinyl, haloalkylsulfinyl, haloalkenylsulfinyl, haloalkynylsulfinyl, alkylsulfonyl, alkenylsulfonyl, alkynylsulfonyl, haloalkyl-sulfonyl, haloalkenylsulfonyl, haloalkynylsulfonyl, alkylamino, alkenylamino, alkynylamino, di(alkyl)amino, di(alkenyl)-amino, di(alkynyl)amino, or trialkylsilyl.

The term “alkylaryl,” as used herein, refers to an aryl group that is bonded to a parent compound through a diradical alkylene bridge, (—CH₂—)ₙ, where n is 1-12 (e.g., n is from 1 to 6) and where “aryl” is as defined above. The term “arylalkyl,” as used herein, refers to an aryl group, as defined above, which is substituted by an alkyl group, as defined above.

The term “alkylcycloalkyl,” as used herein, refers to a cycloalkyl group that is bonded to a parent compound through a diradical alkylene bridge, (—CH₂—)ₙ, where n is 1-12 (e.g., n is from 1 to 6) and where “cycloalkyl” is as defined above.

The term “alkoxy,” as used herein, refers to alkyl-O—, wherein alkyl refers to an alkyl group, as defined above. Similarly, the terms “alkenyloxy,” “alkynyloxy,” and “cycloalkoxy,” refer to the groups alkenyl-O—, alkynyl-O—, and cycloalkyl-O—, respectively, wherein alkenyl, alkynyl, and cycloalkyl are as defined above. Examples of C₁-C₆-alkoxy groups include, but are not limited to, methoxy, ethoxy, C₂H₅—CH₂O—, (CH₃)₂CHO—, n-butoxy, C₂H₅—CH(CH₃)O—, (CH₃)₂CH—CH₂O—, (CH₃)₃CO—, n-pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.

The term “alkylthio,” as used herein, refers to alkyl-S-, wherein alkyl refers to an alkyl group, as defined above. Similarly, the term “cycloalkylthio,” refers to cycloalkyl-S— where cycloalkyl are as defined above.

The term “alkylsulfinyl,” as used herein, refers to alkyl-S(O)—, wherein alkyl refers to an alkyl group, as defined above.

The term “alkylsulfonyl,” as used herein, refers to alkyl-S(O)₂—, wherein alkyl is as defined above.

The terms “alkylamino” and “dialkylamino,” as used herein, refer to alkyl-NH— and (alkyl)₂N— groups, where alkyl is as defined above.

The terms “alkylcarbonyl,” “alkoxycarbonyl,” “alkylaminocarbonyl,” and “dialkylaminocarbonyl,” as used herein, refer to alkyl-C(O)—, alkoxy-C(O)—, alkylamino-C(O)— and dialkylamino-C(O)— respectively, where alkyl, alkoxy, alkylamino, and dialkylamino are as defined above.

The term “heteroaryl,” as used herein, refers to a monovalent aromatic group of from 1 to 15 carbon atoms (e.g., from 1 to 10 carbon atoms, from 2 to 8 carbon atoms, from 3 to 6 carbon atoms, or from 4 to 6 carbon atoms) having one or more heteroatoms within the ring. The heteroaryl group can include from 1 to 4 heteroatoms, from 1 to 3 heteroatoms, or from 1 to 2 heteroatoms. In some examples, the heteroatom(s) incorporated into the ring are oxygen, nitrogen, sulfur, or combinations thereof. When present, the nitrogen and sulfur heteroatoms can optionally be oxidized. Heteroaryl groups can have a single ring (e.g., pyridyl or furyl) or multiple condensed rings provided that the point of attachment is through a heteroaryl ring atom. Preferred heteroaryls include pyridyl, piridazinyl, pyrimidinyl, pyrazinyl, triazinyl, pyrrolyl, indolyl, quinolinyl, isoquinolinyl, quinazolinyl, quinoxalinnyl, furanyl, thiophenyl, furyl, pyrrolyl, imidazolyl, oxazolyl, isoxazolyl, isothiazolyl, pyrazolyl benzofuranyl, and benzothiophenyl. Heteroaryl rings can be unsubstituted or substituted by one or more moieties as described for aryl above.

The term “alkylheteroaryl,” as used herein, refers to a heteroaryl group that is bonded to a parent compound through a diradical alkylene bridge, (—CH₂—)ₙ, where n is 1-12 and where “heteroaryl” is as defined above.

The terms “heterocyclyl,” “heterocyclic” and “heterocyclo” are used herein interchangeably, and refer to fully saturated or unsaturated, cyclic groups, for example, 3 to 7 membered monocyclic or 4 to 7 membered monocyclic; 7 to 11 membered bicyclic, or 10 to 15 membered tricyclic ring systems, having one or more heteroatoms within the ring. The heterocyclyl group can include from 1 to 4 heteroatoms, from 1 to 3 heteroatoms, or from 1 to 2 heteroatoms. In some examples, the heteroatom(s) incorporated into the ring are oxygen, nitrogen, sulfur, or combinations thereof. When present, the nitrogen and sulfur heteroatoms can optionally be oxidized, and the nitrogen heteroatoms can optionally be quaternized. The heterocyclyl group can be attached at any heteroatom or carbon atom of the ring or ring system and can be unsubstituted or substituted by one or more moieties as described for aryl groups above.

Exemplary monocyclic heterocyclic groups include, but are not limited to, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridinyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, triazolyl, triazinyl, and the like.

Exemplary bicyclic heterocyclic groups include, but are not limited to, indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl]or furo[2,3-b]pyridinyl), dihydroisoindolyl, dihydroquinazolinyl (such as 3,4-dihydro-4-oxo-quinazolinyl), tetrahydroquinolinyl and the like.

Exemplary tricyclic heterocyclic groups include carbazolyl, benzidolyl, phenanthrolinyl, acridinyl, phenanthridinyl, xanthenyl, and the like.

The term “alkylheterocyclyl,” as used herein, refers to a heterocyclyl group that is bonded to a parent compound through a diradical alkylene bridge, (—CH₂—)ₙ, where n is 1-12 and where “heterocyclyl” is as defined above. The term “heterocyclylalkyl,” as used herein, refers to a heterocyclyl group, as defined above, which is substituted by an alkyl group, as defined above.

Heretrocyclyl and heteroaryl groups can be unsubstituted or substituted with one or more moieties chosen from alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the disclosure, either unprotected, or protected as necessary, as known to those skilled in the art, for example, as described in Greene, et al., Protective Groups in Organic Synthesis, John Wiley and Sons, Third Edition, 1999

The term “halogen,” as used herein, refers to the atoms fluorine, chlorine, bromine and iodine. The prefix halo- (e.g., as illustrated by the term haloalkyl) refers to all degrees of halogen substitution, from a single substitution to a perhalo substitution (e.g., as illustrated with methyl as chloromethyl (—CH₂Cl), dichloromethyl (—CHCl₂), trichloromethyl (—CCl₃)).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The chemical groups described herein can be unsubstituted or substituted with one or more moieties chosen from alkyl, halo, haloalkyl, hydroxyl, carboxyl, acyl, acyloxy, amino, alkyl- or dialkylamino, amido, arylamino, alkoxy, aryloxy, nitro, cyano, azido, thiol, imino, sulfonic acid, sulfate, sulfonyl, sulfanyl, sulfinyl, sulfamonyl, ester, phosphonyl, phosphinyl, phosphoryl, phosphine, thioester, thioether, acid halide, anhydride, oxime, hydrazine, carbamate, phosphoric acid, phosphate, phosphonate, or any other viable functional group that does not inhibit the biological activity of the compounds of the disclosure, either unprotected, or protected as necessary, as known to those skilled in the art, for example, as described in Greene, et al., Protective Groups in Organic Synthesis, John Wiley and Sons, Third Edition, 1999.

A “Michael acceptor”, as used herein, is an electrophilic moiety that participates in conjugate nucleophilic addition reaction. The Michael acceptor can be an α,β-unsaturated species conjugated to carbonyl or other electron withdrawing moiety. Examples of Michael acceptors include pi-bonds, such as double or triple bonds, conjugated to other pi-bond containing electron withdrawing groups, such as carbonyl, carboxyl, nitro, nitrile, sulfonyl, sulfone, isocyanate, aryl groups, and the like.

A “Michael donor,” as used herein, is a nucleophilic moiety. The Michael donor can also be a functional group containing at least one Michael active hydrogen atom.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

Microparticles

The present disclosure relates to microparticles (also referred to herein as “microgel particles” and “microgels”). The microparticle can comprise heparin or a heparin-like polymer crosslinked to a biocompatible polymer. In some aspects, the heparin or the heparin-like polymer can be directly crosslinked to the biocompatible polymer. In some aspects, the heparin or a heparin-like polymer can be indirectly crosslinked to the biocompatible polymer. For example, the heparin or the heparin-like polymer can be indirectly crosslinked to the biocompatible polymer via a coupling agent. The heparin or a heparin-like polymer can be directly crosslinked to the biocompatible polymer via a Michael type addition reaction. In some examples, the heparin or the heparin-like polymer and the biocompatible polymer can comprise a Michael acceptor or a Michael donor.

Some suitable Michael acceptors include, a maleimide containing group, a vinyl sulfone, vinyl sulfoximine, isocyanate, an acrylate group, a methacrylate group, a styrene group, an acrylamide group, a methacrylamide group, acrylonitrile, a maleate group, a fumarate group, an itaconate group, a vinyl ether group, an allyl ether group, an allyl ester group, a vinyl ester group, a cinnamate group, a cyanoacrylate, a vinyl ketone, a nitro ethylene, a α,β-unsaturated aldehyde, a vinyl phosphonate, a vinyl pyridine, an azo compound, a β-keto acetylene, an acetylene ester, or combinations thereof.

Suitable examples of Michael donor include, but are not limited to, thiols, amines, hydroxyls, phosphorous containing groups, malonate esters, acetoacetate esters, malonamides, acetoacetamides, cyanoacetate esters, cyanoacetamides, or combinations thereof.

As described, the microparticles disclosed herein can contain heparin or a heparin-like polymer. Heparin is a heterogenous group of straight-chain anionic mucopolysaccharides, called glycosaminoglycans. The heparin used in the described microparticle can be a high molecular weight heparin (HMWH) or a low molecular weight heparin (LMWH). Heparin can have a molecular weight of from about 1,000 to about 50,000 Daltons, for example, from about 1,000 to about 20,000 Daltons. In some examples, the heparin used in the described microparticle is a low molecular weight heparin (LMWH). LMWH refers to heparin with molecular weights of from about 1,000 to about 10,000 Daltons. For example, the molecular of the LMWH can be about 10,000 Daltons or less, about 9,500 Daltons or less, about 9,000 Daltons or less, about 8,500 Daltons or less, about 8,000 Daltons or less, about 7,500 Daltons or less, about 7,000 Daltons or less, about 6,500 Daltons or less, or about 6,000 Daltons or less. Synthetic or natural heparin can be used in the microparticles. “Heparin-like polymers”, as used herein, refers to a polysaccharide with at least one negative charge per two saccharide rings and no more than one positive charge per ten saccharide rings, under the general conditions of a cell culture medium such as pH and temperature In some examples, the heparin-like polymers can bind to protein growth factors. The heparin-like polymer can have a molecular weight of from about 1,000 to about 50,000 Daltons. For example, the molecular of the heparin-like polymer can be about 20,000 Daltons or less, about 15,000 Daltons or less, about 10,000 Daltons or less, or about 7,500 Daltons or less. Synthetic or natural heparin-like polymers can be used in the microparticles. Suitable examples of heparin-like polymers can include dextran sulfate, chondroitin sulfate, heparin sulfate, fucan, alginate, copolymers thereof, and blends thereof.

The heparin or heparin-like polymer can be functionalized to react directly or indirectly with the biocompatible polymer. In some examples, the heparin or heparin-like polymer can comprise a Michael donor or a Michael acceptor. For example, the heparin or heparin-like polymer can comprise a maleimide containing group. Functionalized heparin or heparin-like polymer are commercially available or can be obtained as disclosed herein.

The microparticles can also contain a biocompatible polymer. In some examples, the biocompatible polymer is biodegradable. “Biocompatible” and “biologically compatible”, as used herein, generally refer to polymers that are, along with any metabolites or degradation products thereof, generally non-toxic to cells and tissues, and which do not cause any significant adverse effects to cells and tissues when cells and tissues are incubated (e.g., cultured) in their presence.

Suitable biocompatible polymers can include polyalkylene oxide, polylactic acid, polyacrylic acid, polyurethane, polyphosphazene, polysaccharide, dextran, polyvinyl pyrrolidone, polyvinyl alcohol, polyacrylamide, copolymers thereof, and blends thereof. In some examples, the biocompatible polymer includes a polyalkylene oxide. The biocompatible polymer such as polyalkylene oxide can be a multi-arm polyalkylene oxide having from 3 to about 10 arms. For example, the multi-arm polyalkylene oxide can have 3, 4, 5, 6, 7, 8, 9, or 10 arms. In some examples, the multi-arm polyalkylene oxide includes a four arm polyethylene oxide.

The biocompatible polymer can be functionalized to react with heparin or the heparin-like polymer directly or indirectly. In some examples, the biocompatible polymer can comprise a Michael donor. For example, the biocompatible polymer can comprise a thiol. Suitable biocompatible polymers can include PEG-maleimide, PEG-thiol, PEG-amine, PEG-vinylsulfone, PEG-orthopyridyl disulfide, PEG-isocyanate, PEG-succimidyl succinate, PEG-silane, PEG-acrylate, PEG-hydrazide, PEG-tresylate, PEG-propion aldehyde, PEG-tosylate, PEG-succimidyl glutarate, PEG-nitrophenyl carbonate, blends thereof, copolymers thereof. Biocompatible polymers are commercially available, for example, from Sigma Aldrich, Shearwater Polymers (Huntsville, Ala.) and Texaco Chemical Co. (Houston, Tex.).

The biocompatible polymer can have a molecular weight of from about 1,000 to about 50,000 Daltons. For example, the molecular weight of the biocompatible polymer can be from about 1,000 to about 30,000 Daltons, from about 1,000 to about 25,000 Daltons, or from about 1,000 to about 20,000 Daltons. In some examples, the molecular weight of the biocompatible polymer can be about 20,000 Daltons or less, about 15,000 Daltons or less, about 12,000 Daltons or less, about 10,000 Daltons or less, about 8,000 Daltons or less, or about 7,000 Daltons or less.

In some examples, the heparin or a heparin-like polymer can be indirectly attached to the biocompatible polymer. For example, the heparin or a heparin-like polymer can be indirectly attached to the biocompatible polymer via a Michael type addition reaction. The method for indirectly attaching the heparin or a heparin-like polymer to the biocompatible polymer can be via a coupling agent. The coupling agent can comprise a Michael donor or a Michael acceptor.

The coupling agent can contain two or more functional groups which are able to react with functional groups on the heparin or heparin-like polymer and functional groups on the biocompatible polymer. For example, the coupling agent can contain nucleophilic groups which react with electrophilic groups found in the heparin or heparin-like polymer or vice versa. In some examples, the coupling agent can have a structure represented by Formula I:

(A)ₚ(R)(D)_{q} Formula I

wherein A is a bond or a moiety that can form a bond with the heparin or the heparin-like polymer, D is a bond or a moiety that can form a bond with the biocompatible polymer, R is a linker for A and D, and p and q are integers from 1 to 25. In some examples, p and q can independently be an integer with a value of 1, 2, 3, 4, 5, 6, 7, 8, 9, 10, 11, 12, 13, 14, 15, 16, 17, 18, 19, 20, 21, 22, 23, 24, or 25.

The moieties A and D in the coupling agent can contain a Michael acceptor and/or a Michael donor, depending on the functional group on the heparin or heparin containing polymer and the biocompatible polymer. A and D, for each occurrence, can independently include a Michael acceptor, Michael donor, a group reactive with an amine, a group reactive with a carboxylic acid, a group reactive with a thiol, a group reactive with a hydroxyl, a group reactive with an ester, a group reactive with an carbonyl, a group reactive with a carbon-carbon unsaturated bond, or combinations thereof. For example, in some examples, the coupling agent can comprise a Michael acceptor and an amino group. It is to be understood that when the coupling agent is linked to the heparin or the heparin-like polymer and the biocompatible polymer, A and D represent a moiety bonded to the heparin-like polymer and the biocompatible polymer, respectively. For example, where A is described as an amine containing group in the coupling agent, when the coupling agent is bonded to the heparin or heparin-like polymer, A is then defined as the result of a reaction between the amine containing group and the heparin or heparin-like polymer (e.g., a secondary or tertiary amine or an amide). Similarly, when D is described as a Michael acceptor in the coupling agent, when the coupling agent is bonded to the biocompatible polymer, D is then defined as the result of a reaction between the Michael acceptor and a Michael donor (e.g., a an alkyl, thio ether, ether, or amine beta to a carbonyl).

The linker, R, in the coupling agent can be an atom such as oxygen, sulfur, carbon, boron, or nitrogen. The linker can also be a radical such as a substituted or unsubstituted alkoxy, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heterocycloalkyl, substituted or unsubstituted heteroaryl, substituted or unsubstituted ether, or substituted or unsubstituted amine. In some examples, the linker can be a polymer. The polymer is suitable for increasing the distance between the reactive functional groups at the termini The linker can also be one or more atoms that separate moieties A and D.

Suitable coupling agents include, but are not limited to, amino alkyl maleimide (such as N-(2-aminoethyl) maleimide. One of ordinarily skill in the art will also recognize that other coupling agents, with different number of atoms, can be used. Coupling agents are commercially available, for example, from Sigma Aldrich and VWR.

The microparticle can have an average particle size of from about 2 μm to about 30 μm. For example, the average particle size of the microparticle can be from about 2 μm to about 25 μm, from about 2 μm to about 20 μm, from about 5 μm t about 30 μm, or from about 10 μm to about 30 μm. In some examples, the average particle size of the microparticle can be about 5 μm or greater, about 7 μm or greater, about 10 μm or greater, about 12 μm or greater, about 15 μm or greater, about 20 μm or greater. In some examples, the average particle size of the microparticle can be about 30 μm or less, about 25 μm or less, about 20 μm or less, or about 15 μm or less.

Compositions comprising the microparticles disclosed herein are also described. The composition can contain a plurality of microparticles. In some examples, the composition can be a cell culture medium comprising a plurality of microparticles. The cell culture medium can comprise cell culture ingredients suitable for the maintenance of eukaryotic cells. For example, the cell culture medium can comprise a protein growth factors or peptide fragment thereof.

The cell culture medium can also include trace elements such as Zn, Ca, Mn, Ni, Al, Cr and Se, initially provided as the ions Zn²⁺, Ca⁺, Mn²⁺, Ni²⁺, Al³⁺, and Cr³⁺. The trace elements may comprise Mn (about 20 nM), Cr (about 20 nM), Zn (about 20 μM), Ni (about 4 nM), Co (about 4 nM), Cu (about 0.4 μM), Al (about 4 nM) and Se (about 200 nM). Sterols may be included. Two or more of cholesterol, campesterol, desmosterol, ergosterol, fucosterol, β-sitosterol, stigmasterol or metabolically acceptable derivatives thereof—may be used. Other metabolically acceptable derivatives may be used, i.e. derivatives which perform the role of such sterols in cell culture satisfactorily and without toxic effects.

Methods

Methods of making the described microparticles are also disclosed. The method can include providing a first solution of heparin or a heparin-like polymer and a second solution of the biocompatible polymer. Providing the first solution and the second solution can include dissolving the heparin or a heparin-like polymer and the biocompatible polymer in a first solvent and a second solvent, respectively. The first solvent and a second solvent can be a buffer of pH from 6.5 to 8.5. For example, the pH can be from 6.5 to 8 or 7 to 8. In some examples, the pH can be 6.5, 7, 7.2, 7.4, 7.5, 7.6, 7.7, 7.8, 7.9, 8, or 8.5, where any of these values can be an upper or lower endpoint of a range. In some examples, the first solvent and a second solvent can be the same. The first solution and the second solution can be mixed to form a mixture.

Mixing the heparin or heparin-like polymer and the biocompatible polymer can include reacting the heparin or heparin-like polymer and the biocompatible polymer through the use of the coupling agent. The coupling agent can be bonded to the heparin or heparin-like polymer first, though moiety A in Formula 1. Then the biocompatible polymer can be bonded to the conjugate of the heparin or heparin-like polymer with the coupling agent via moiety D in Formula 1. An alternative process can be used where the biocompatible polymer is first bonded to the coupling agent through moiety D in Formula 1. Then moiety A can be used to bond to the heparin or heparin-like polymer. In still another alternative process, the heparin or heparin-like polymer, biocompatible polymer, and coupling agent can be simultaneously added so that the heparin or heparin-like polymer reacts with and forms a bond through moiety A of the coupling agent and the biocompatible polymer reacts with and forms a bond through moiety D of the coupling agent. In specific examples, The reaction of the two polymers with the coupling reagent can be a Michael addition reaction, nucleophilic substitution, electrophilic substitution, condensation reaction, or combinations thereof.

In some examples, the method can comprise coupling the heparin or heparin-like polymer to a coupling agent represented by Formula I:

(A)p(R)(D)q Formula I

wherein

A is a bond or a moiety that can react with the heparin or the heparin-like polymer,

D is a bond or a moiety that can react with the biocompatible polymer,

R is a linker for A and D, and

p and q are integers from 1 to 25. The heparin or heparin-like polymer and the coupling agent can be coupled before mixing with the first solution and the second solution. Coupling the heparin or heparin-like polymer to the coupling agent can include reacting the heparin or heparin-like polymer and the coupling agent via a Michael addition reaction, nucleophilic substitution, condensation reaction, hydrolysis, or combinations thereof. In some examples, coupling the heparin or heparin-like polymer to the coupling agent can include a condensation reaction between A and the heparin or heparin-like polymer.

The biocompatible polymer and the coupling agent can be coupled before mixing with the first solution and the second solution. Coupling the bicompatible polymer to the coupling agent can include reacting the heparin or heparin-like polymer and the coupling agent via a Michael addition reaction, nucleophilic substitution, electrophilic substitution, condensation reaction, or combinations thereof. In some examples, coupling the biocompatible polymer to the coupling agent can include a Michael addition reaction between D and the biocompatible polymer.

An oil-in-water emulsion of the mixture can be formed by adding the mixture to an oil and/or a surfactant. Suitable oil can include paraffin oil, squalane, pristane, polyisobutene oil, hydrogenated polyisobutene oil, polydecene oil, polyisoprene oil, polyisopropene oil, and combinations thereof. Suitable surfactants can include Span, Tween, Brij, and combinations thereof. The Span surfactants can include sorbitan fatty acid esters, while the Tween surfactants can include ethoxylated sorbitan fatty acid esters. The Brij surfactants can include ethoxylated fatty alcohols. Representative examples of surfactants can include Span, Span 20, Span 40, Span 60, Span 80, Tween 20, Tween 40,

Tween 60, Tween 80, Tween 85, Brij 35, or combinations thereof. One of ordinary skill in the art knows how to adjust the ratio of the surfactant and oil so the hydrophilic-lipophilic balance is within the water-in-oil emulsion range. The method can also include homogenizing the oil-in-water emulsion. Homogenizing can be carried out from about 2,000 rpm to 5,000 rpm for 10 minutes or less. For example, homogenizing can be carried out at 3,000 rpm for 3 minutes or less.

Methods of using the disclosed microparticles are also disclosed. The microparticles can encapsulate, be mixed with, or be ionically or covalently coupled to any of a variety of therapeutic, prophylactic or diagnostic agents or other protein bio-active agents, thus making them suitable for controlled release. Examples of suitable therapeutic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, mRNAs which can inhibit transcription, and ribozymes. Representative examples of suitable materials which can be encapsulated include antibodies, receptor ligands, enzymes, adhesion peptides, saccharides and polysaccharides, blood clotting factors, inhibitors or clot dissolving agents such as streptokinase and tissue plasminogen activator, antigens for immunization, and hormones and growth factors. The microparticles can also be used to encapsulate cells and tissues. Compounds with a wide range of molecular weight can be encapsulated, for example, between 100 and 500,000 grams or more per mole. In some examples, the microparticles can be used in cell culture media and other biological assays. For example, the microparticles disclosed herein can be used for controlled release of cell growth factors in tissue engineering.

In some examples, the microparticles can be used in medical devices for use in biodegradable implants. For example, the biocompatible polymer can be a degradable polymer which is useful for preparing a variety of medical devices, including but not limited to, biodegradable implants, sutures, matrices and scaffolds for drug delivery and/or tissue engineering. Devices prepared from the microparticles can include particles, matrices and/or scaffolds for delivery of a therapeutic or diagnostic agent, particularly for controlled release of an active agent, particles, matrices and/or scaffolds for tissue engineering, cell encapsulation, targeted delivery, guided tissue regeneration; and diagnostics.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Synthesis of Heparin-maleimide Microgels

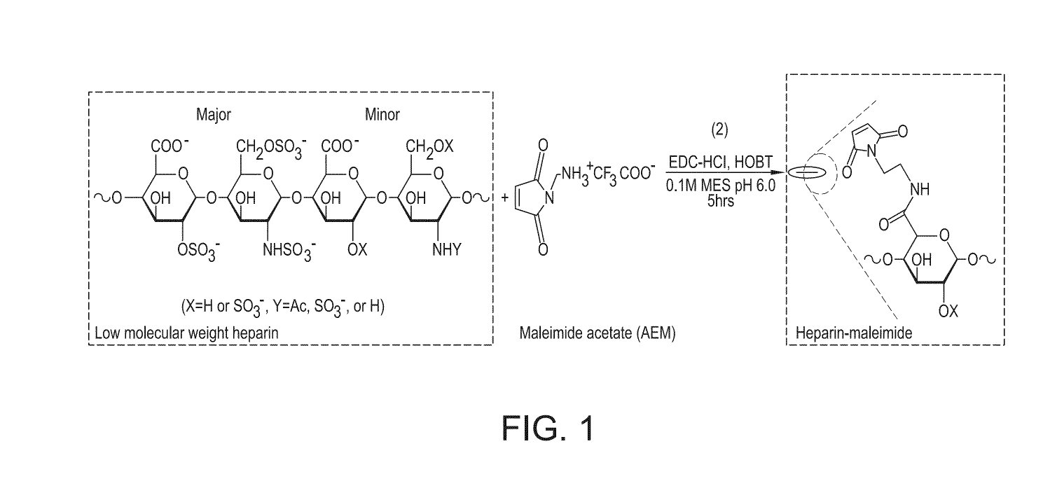

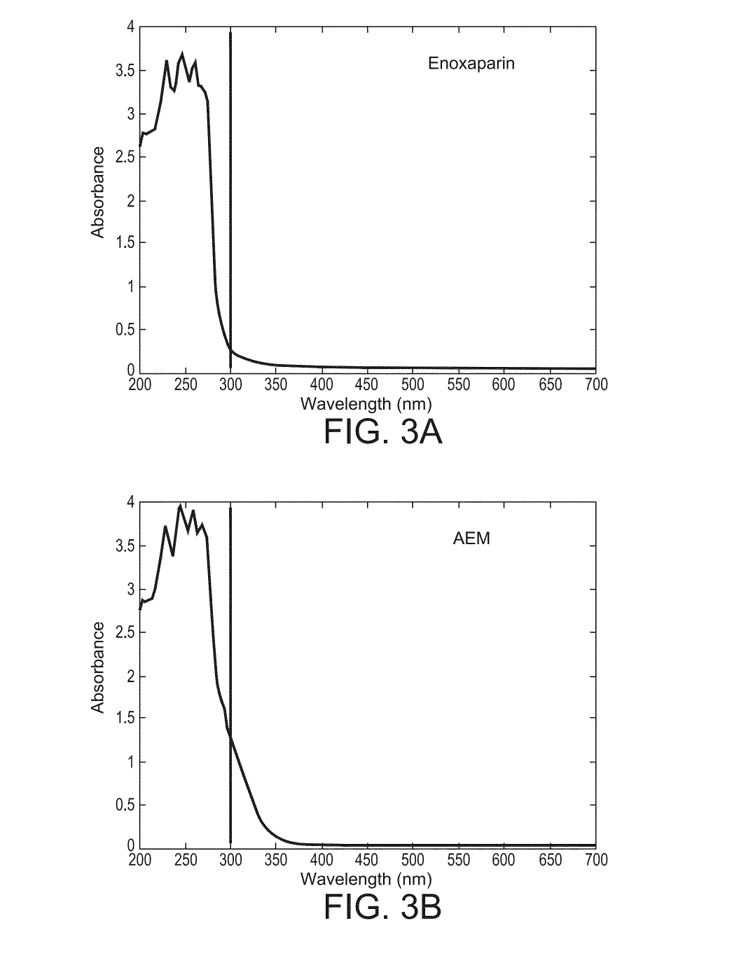

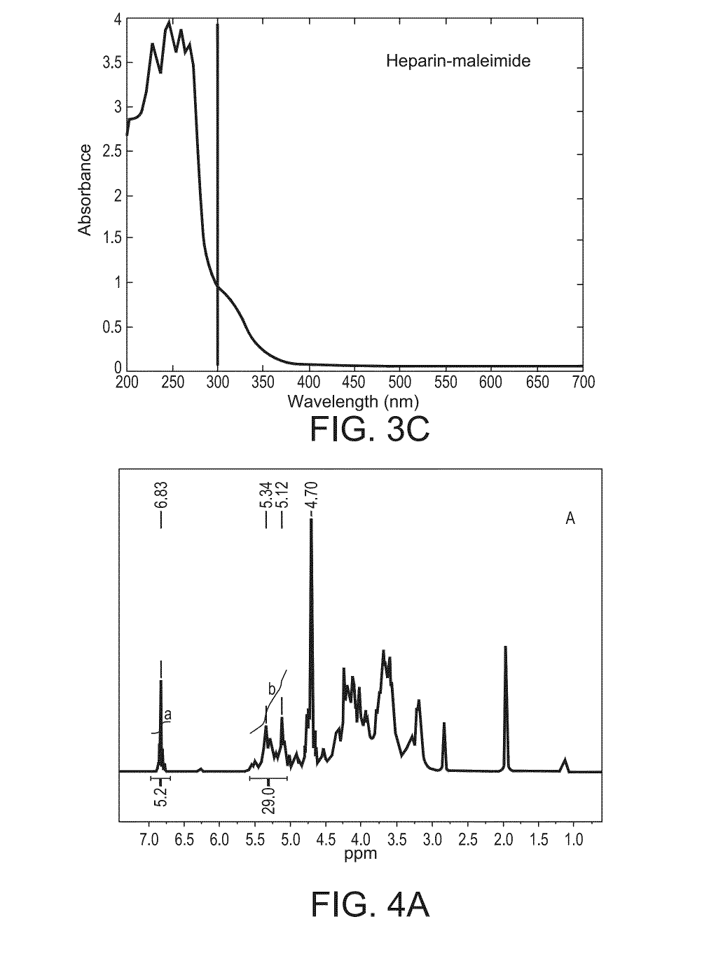

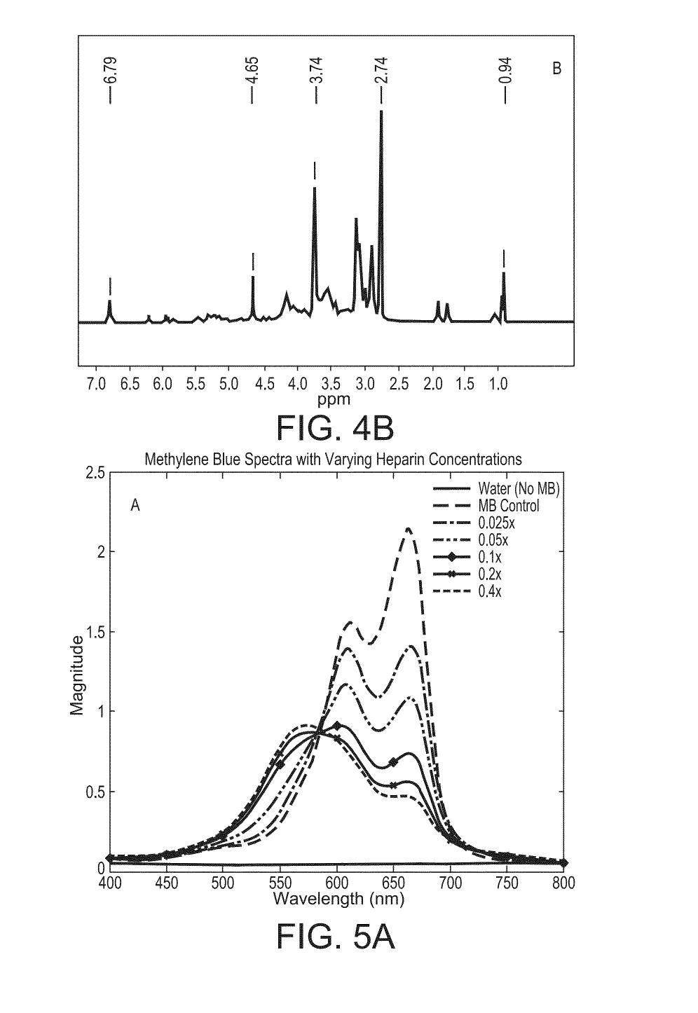

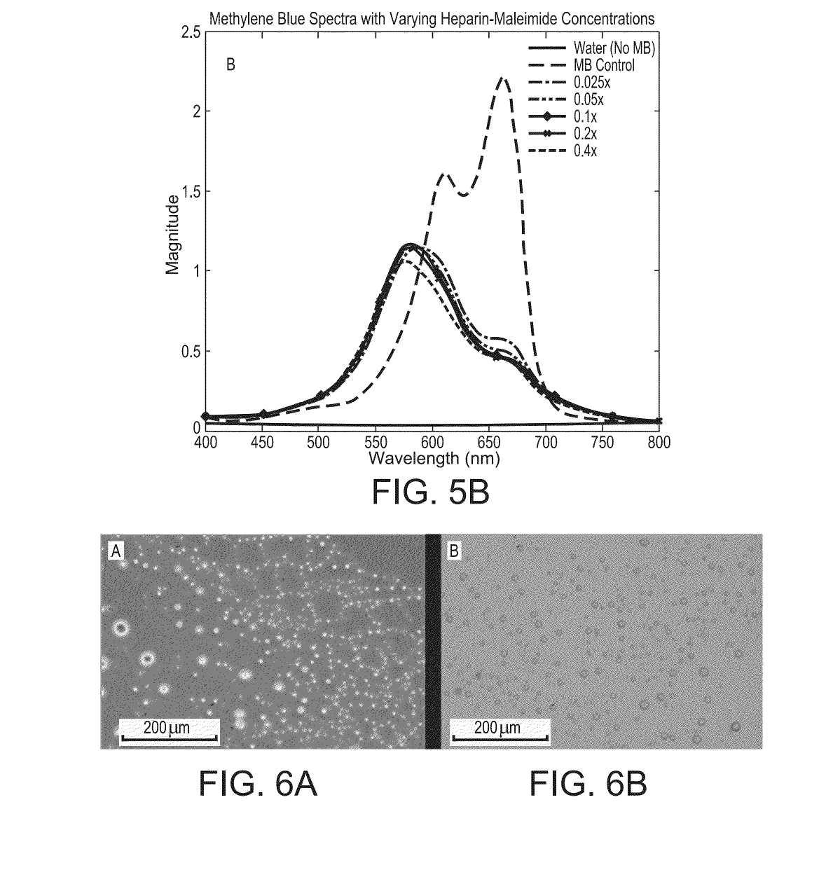

Synthesis of heparin-maleimide compounds (Mal-LMWH): 500 mg LMWH (0.06 mmol) was dissolved with 103 mg 1-hydroxybenzotriazole hydrate (0.67 mmol), 103 mg maleimide acetate (0.67 mmol) and 103 mg N-(3-Dimethylaminopropyl)-N0-ethylcarbodiimide hydrochloride (0.54 mmol) dissolved in 50 mL of 0.1 M MES pH 6.0. FIG. 1 is an illustration of the synthetic scheme of Mal-LMWH as described in Baldwin et al., Polm. Chem., 2013, 4:133-143. The reaction proceeded overnight at room temperature with stirring. The product was purified by dialysis (MWCO 1000) against 4 L of 1M NaCl solution and then subsequently against de-ionized water each with 4 volumes exchanges over 24 h. The starting materials and the heparin-maleimide sample were characterized via UV/VIS (FIG. 3A through FIG. 3C). The sample was also characterized via ¹H NMR indicating a degree of functionalization of 2.6. The ¹H NMR spectrum of the heparin-maleimide is shown in FIG. 4B. The starting materials and the freeze-dried sample were characterized via UV/VIS spectroscopy with methylene blue (FIG. 5A and FIG. 5B).

### Example 2

### Synthesis of Functionalized PEG (PEG-4-thiol)

The synthesis of thiolated four-arm PEG can be carried out as described in Baldwin et al. (Polm. Chem., 2013, 4:133-143). PEG (1 meq.), mercaptoacid (40 meq. MP, MIB, MPP or DMMPP) and PTSA (0.4 meq.) are dissolved in toluene. Under a flow of nitrogen, the reaction is refluxed with stirring for 48 h. Water is collected by using a Dean Stark trap. Toluene is remove under reduce pressure and the polymer is precipitated 3 times in cold ether. The polymer is reduced by dissolving 1 meq. polymer in methanol with DTT (1 meq.) and triethylamine (1 meq.) under nitrogen for 5 hours. The reaction mixture is acidified with trifluoroacetic acid (1.1 meq.), and the polymer is precipitated in ether and rinsed with 2-propanol then hexane. The products are stored under argon or vacuum at room temperature to maintain the reduced thiol during storage.

### Example 3

### Microgel Formation

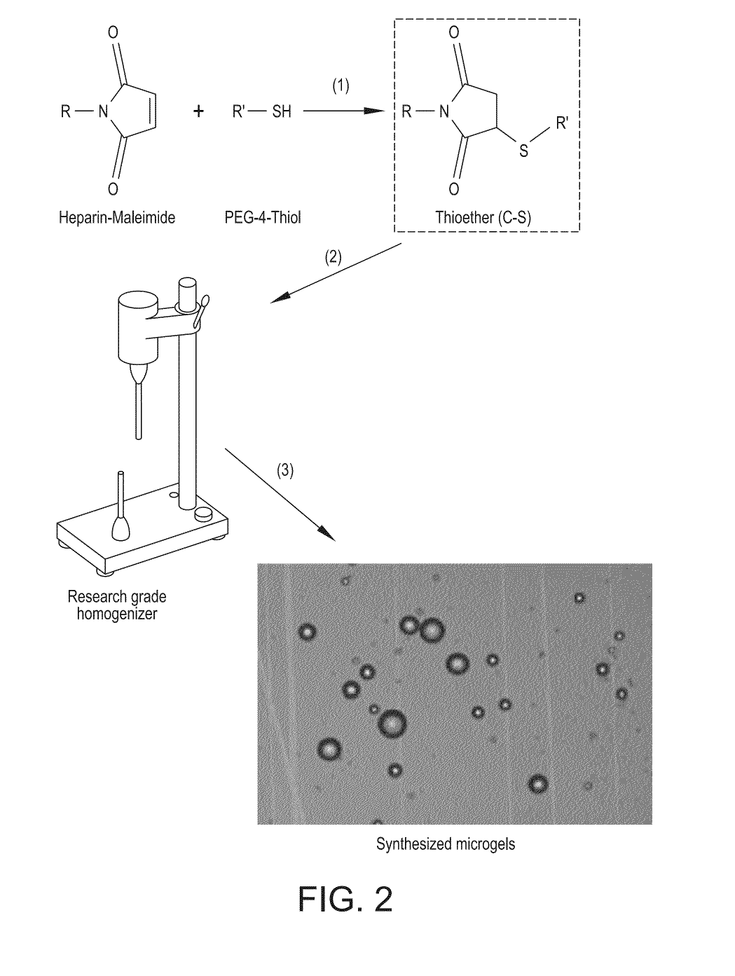

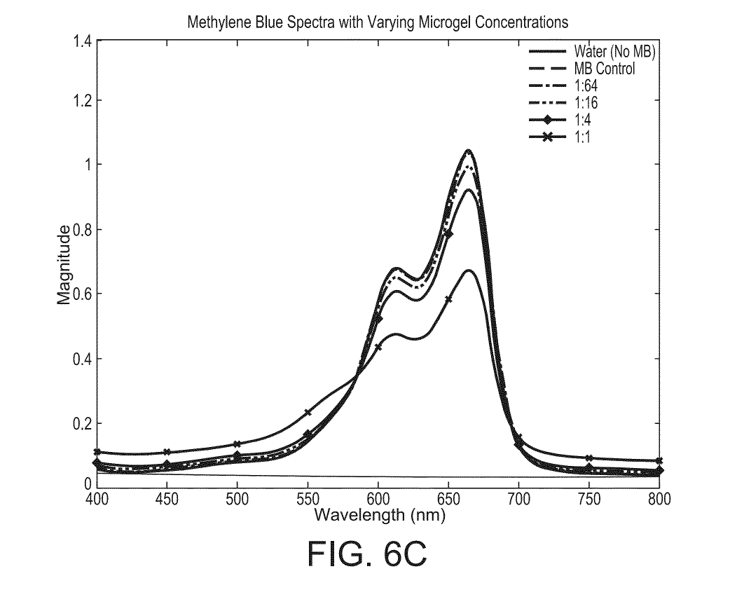

Heparin-maleimide-PEG microgels were prepared as described in FIG. 2. The microgels were synthesized using a Michael addition cross-linking during (water-in-oil) emulsion (MADE), wherein the cross-linking occurs through a Michael-addition reaction within surfactant stabilized aqueous droplets to form microgels. Equimolar amounts (PEG-4-thiol:heparin maleimide) of the functionalized PEG and heparin-maleimide were dissolved separately into 0.3 M triethanolamine buffer at pH 7.8 to a total combined concentration of 60% (w/v), in a total volume of 100 μL. The two solutions were combined, mixed, and added to 15 mL of paraffin oil with 1% (v/v) surfactant (Span 80/Tween 80 combination to achieve an HLB=5). The entire mixture was then immersed in a hot water bath at 40-45° C. and homogenized for 3 min at 3000 rpm using a homogenizer. The resulting surfactant-stabilized water-in-oil emulsion was incubated for at least 2 h at 37° C. to allow the cross-linking reaction to complete, forming solid, cross-linked, micro-sized hydrogels (microgels). Thus, as the reaction proceeds, the mixture becomes a suspension of solid microgels in oil, rather than an emulsion. These microgels were removed from oil, residual surfactant, and un-reacted material by a series of centrifugal washes with fresh oil and water. Microgels were centrifuged at 10,000×g for 20 min, supernatant was discarded, re-suspended in deionized water, and vortexed. The microgel particles were characterized via UV/VIS spectroscopy and light microscopy (FIG. 6A through FIG. 6C).

Size and morphology: Microgels were sized in water (swollen state) or paraffin oil (relaxed state) by imaging on a hemocytometer. Particle size and distribution were obtained from ImageJ. To observe morphology, the microgels were dried at low vacuum for 48 h, or lyophilized for 24 h, and analyzed via SEM. They were also imaged under light microscopy during the hydrolytic degradation studies (detailed in Section 3.2) and analyzed using the ImageJ particle analysis toolkit (public domain image processing and analysis software from the National Institutes of Health) for sizing.

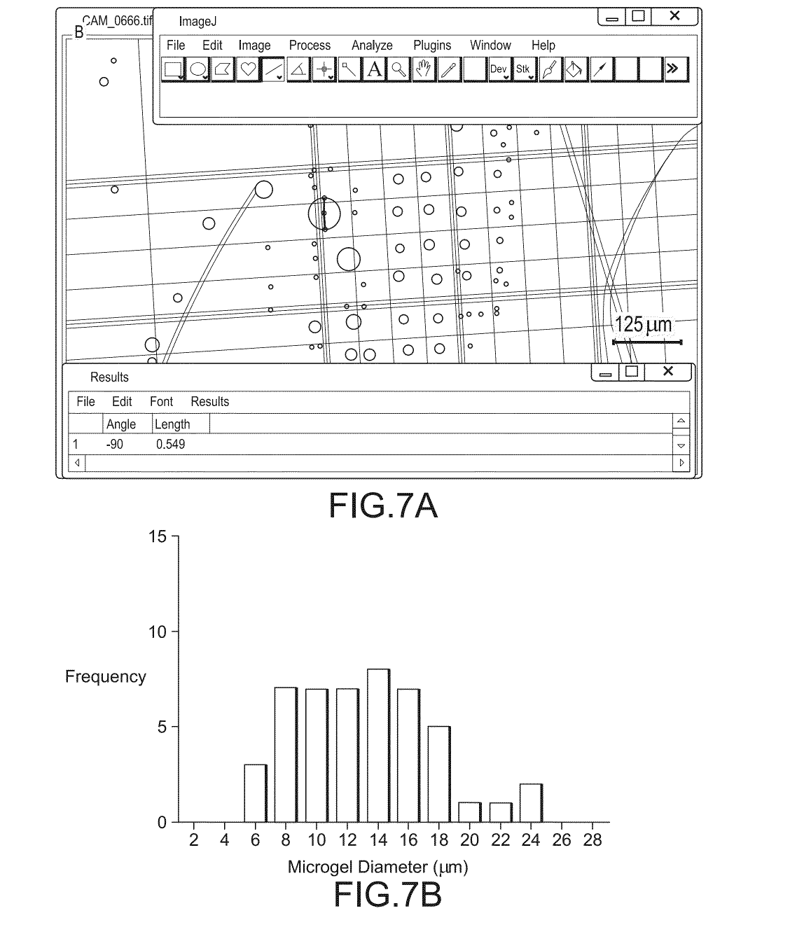

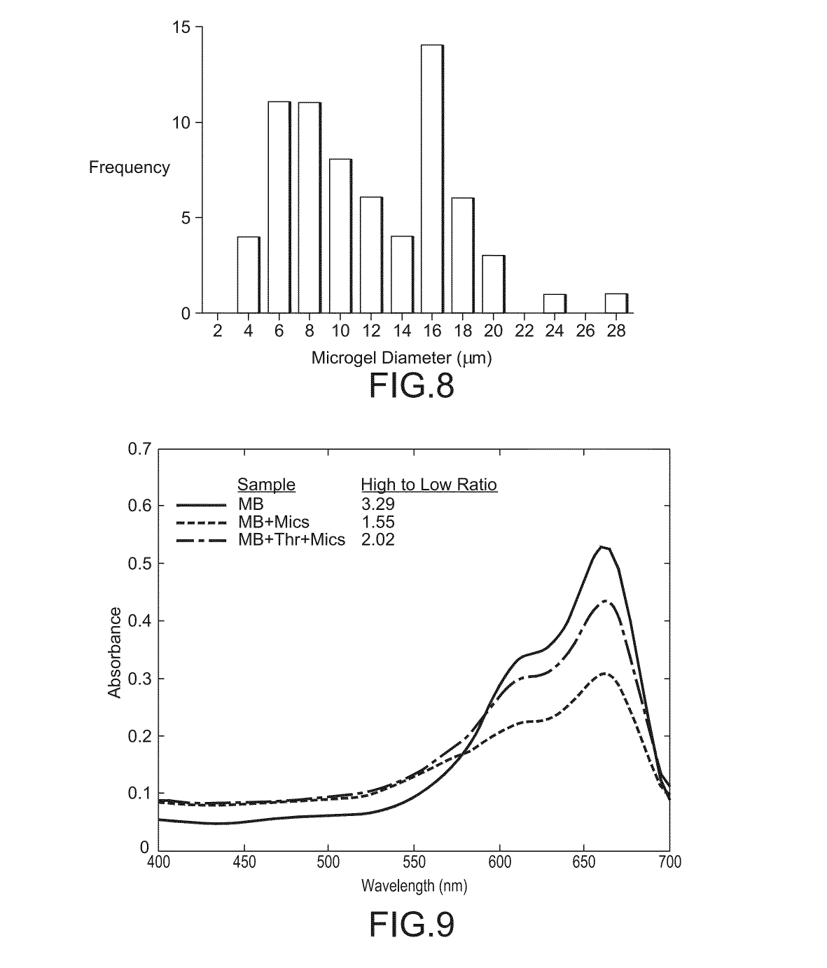

Particle size and particle size distribution of the microgel particles were obtained for particles synthesized in TEA buffer, pH 7.8 (FIG. 7B) and in TEA buffer, pH 7.5 (FIG. 8).

### Example 4

### Thrombin Binding Assay

Thrombin was used to demonstrate binding efficacy to a growth factor-like protein. Thrombin was used because its molecular weight (36,700 Da) is similar to common growth factors such as VEGF, IGF, and FGF. Thrombin also contains a positive charge which is necessary for electrostatic interaction with heparin. A concentration of 100 nM was used. Three samples, (a) methylene blue and water, (b) methylene blue and microgel, and (c) methylene blue, microgel, and thrombin were prepared. The samples were characterized via UV/VIS spectroscopy (FIG. 9). The data were analyzed quantitatively by taking the numerical integration around two peaks, 550-570 nm and 660-680 nm.

Methylene blue bound to heparin has a characteristic shift when compared to methylene blue in solution. There is a slight restoration of the high peak when thrombin was added. This suggests thrombin is displacing some of the bound methylene blue.

### Example 5

### Microgel Binding Capacity

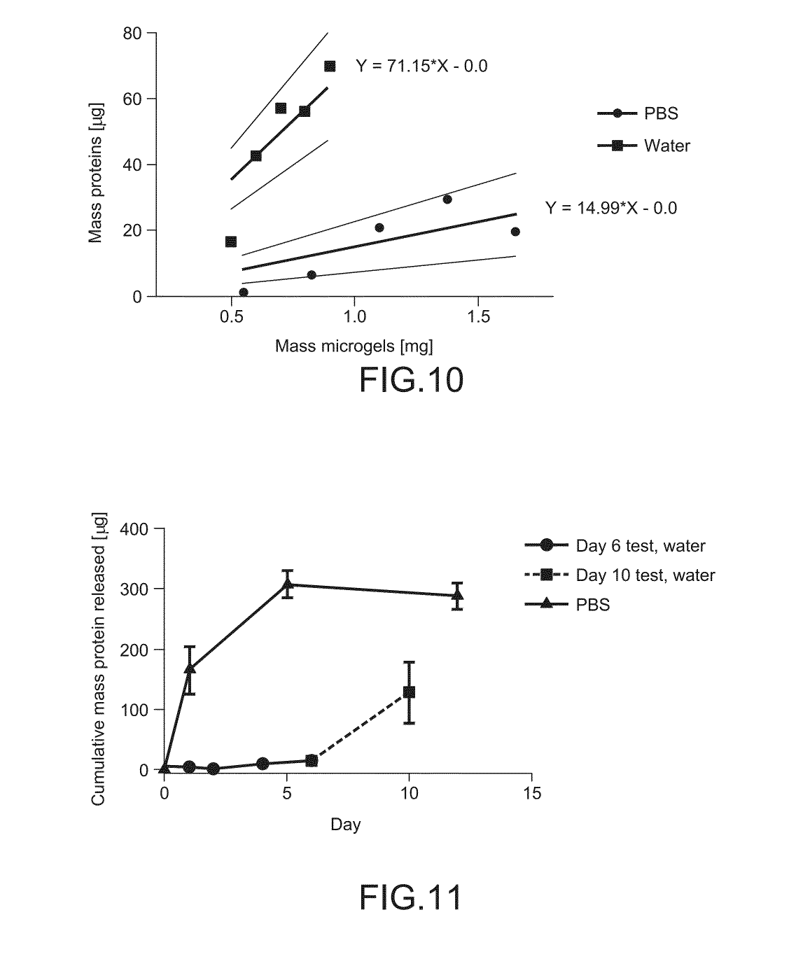

Different concentrations of microgels were resuspended with an excess of thrombin in either PBS or water and incubated at room temperature until a binding equilibrium was reached. Unbound protein was removed by centrifugation and aspiration. The amount of protein bound was measured using a standard BCA assay (FIG. 10).

The microgels have a binding capacity to positively charged proteins of roughly 10-70 μg protein per mg of microgels. The binding efficiency was found to depend on the protein used and the salt content of the solution. In the case of thrombin, binding efficiency was found to be lower in salt-containing solutions, but still on the same order of magnitude as the efficiency in water.

### Example 6

### Glutathione-Mediated Protein Release

Microgel degradation and subsequent protein release were measured by incubating the microgels with 10 mM glutathione (GSH), which is known to reduce disulfide (S—S) and thioether (C—S—C) bonds. The microgels were resuspended with GSH in either water or PBS. The mass of protein released was measured by taking supernatant samples at different time points and employing a standard BCA assay (FIG. 11).

In water, significant protein release was only observed between days 6 and 10, whereas in PBS, degradation started on day 1. In PBS, nearly all protein was released by day 5. These data show that the microgels do have the capacity to release sequestered protein in the presence of GSH.

### Example 7

### Microgel Cytotoxicity

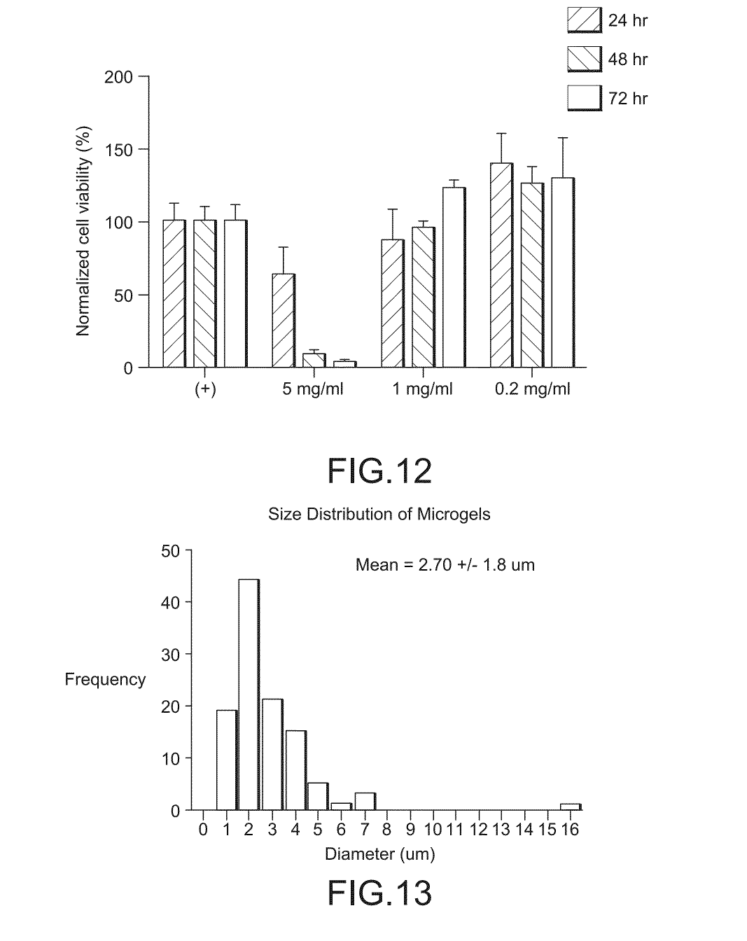

Cell viability of 3T3 cells cultured with microgels in normal media was measured using a standard MTS assay. Metabolic enzymes that are active in viable cells reduce the MTS dye and produce a purple colored formazan product that can be detected with a standard plate reader. Viability was measured at 24, 48, and 72 hour time points. (FIG. 12)

The microgels are not cytotoxic to 3T3 cells at concentrations of 1 mg/mL and lower. There was a significant cytotoxic effect of our microgels at the 5 mg/mL concentration. The final microgel product should be used at a concentration lower than 1 mg/mL to avoid undesired cell effects.

### Example 8

### Size Characterization

Microgels were resuspended in water and placed on a standard hemocytometer. Digital images were captured and analyzed in ImageJ. The ImageJ measuring tool was used to estimate diameters of 109 microgels from 9 different images. The mean diameter is 2.70+/−1.8 μm. The vast majority of microgels are between 1 and 5 μm in diameter (FIG. 13).

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A microparticle, comprising: heparin or a heparin-like polymer coupled to a biocompatible polymer, wherein the microparticle has an average diameter of from about 2 to about 30 μm.

2. The microparticle of claim 1, wherein the heparin or the heparin-like polymer is coupled to the biocompatible polymer by a coupling agent.

3. The microparticle of claim 1, wherein the coupling agent is represented by Formula I:
(A)ₚ(R)(D)_{q} Formula I
wherein
A is a bond or a moiety which is bonded to the heparin or the heparin-like polymer,
D is a bond or a moiety which is bonded to the biocompatible polymer,
R is a linker for A and D,
p and q are integers from 1 to 25.

4. The microparticle of claim 3, wherein A, for each occurrence, independently includes a moiety bonded to the heparin or the heparin-like polymer.

5. The microparticle of claim 3, wherein the moiety bonded to the heparin or the heparin-like polymer is formed from a Michael addition reaction, nucleophilic substitution, electrophilic substitution, condensation reaction, or combinations thereof.

6. The microparticle of claim 3, wherein the moiety bonded to the heparin or the heparin-like polymer is formed from a condensation reaction with the heparin or heparin-like polymer.

7. The microparticle of claim 3, wherein D, for each occurrence, independently includes a moiety bonded to the biocompatible polymer.

8. The microparticle of claim 3, wherein the moiety bonded to the biocompatible polymer is formed from a Michael addition reaction, nucleophilic substitution, condensation reaction, hydrolysis, or combinations thereof.

9. The microparticle of claim 3, wherein the moiety bonded to the biocompatible polymer is formed from a Michael addition reaction with the biocompatible polymer.

10. The microparticle of claim 3, wherein the linker R includes oxygen, sulfur, carbon, boron, nitrogen, substituted or unsubstituted alkoxy, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heterocycloalkyl, substituted or unsubstituted heteroaryl, substituted or unsubstituted ether, substituted or unsubstituted amine, and a polymer.

11. The microparticle of claim 1, wherein the heparin or heparin-like polymer has a molecular weight of from about 1,000 to about 50,000 Daltons.

12. The microparticle of claim 1, wherein the heparin-like polymer is a polysaccharide having at least one negative charge per two saccharide rings and no more than one positive charge per ten saccharide rings.

13. The microparticle of claim 1, wherein the heparin-like polymer is selected from the group consisting of dextran sulfates, chondroitin sulfates, heparin sulfates, fucans, and alginates.

14. The microparticle of claim 1, wherein the biocompatible polymer has a molecular weight of from about 1,000 to about 30,000 Daltons.

15. The microparticle of claim 1, wherein the biocompatible polymer includes polyalkylene oxide, polylactic acid and derivatives thereof, polyacrylic acid and derivatives thereof, polyurethane, polyphosphazene, polysaccharide, dextran, polyvinyl pyrrolidone, polyvinyl alcohol, polyacrylamide, copolymers thereof, and blends thereof.

16. The microparticle of claim 1, wherein the biocompatible polymer comprises a polyalkylene oxide.

17. The microparticle of claim 16, wherein the polyalkylene oxide is a multi-arm polyalkylene oxide having from 3 to 10 arms.

18. The microparticle of claim 17, wherein the multi-arm polyalkylene oxide has 4 arms.

19. A composition comprising a plurality of microparticles of claim 1.

20. A cell culture medium comprising a microparticle or composition of claim 1 and a protein growth factor or peptide fragment thereof having a domain that binds heparin or heparin-like polymer.

21. The cell culture medium of claim 20, wherein the growth factor or peptide fragment thereof is selected from the group consisting of neurturin persephin, IGF-1A, IGF-1β, EGF, NGFβ, NT-3, BDNF, NT-4, TGF-β3, and TOF-β4.

22. A method of making a microparticle, comprising:
mixing a first solution of the heparin or the heparin-like polymer and a second solution of the biocompatible polymer, to thereby form a mixture;
adding the mixture to an oil and a surfactant and homogenizing to form a water-in-oil emulsion;
thereby forming the microparticle.

23. The method of claim 22, where the pH of the mixture is from 5 to 9.

24. The method of claim 22, where the mixture is homogenized from about 2,000 rpm to 5,000 rpm for 10 minutes or less.

25. The method of claim 22, wherein the oil is selected from the group consisting of paraffin oil, squalane, pristane, polyisobutene oil, hydrogenated polyisobutene oil, polydecene oil, polyisoprene oil, polyisopropene oil, and combinations thereof.

26. The method of claim 22, wherein the surfactant is selected from the group consisting of Span, Span 20, Span 40, Span 60, Span 80, Tween 20, Tween 40, Tween 60, Tween 80, Tween 85, Brij 35, and combinations thereof.

27. The method of claim 22, further comprising combining the microparticle with a therapeutic, prophylactic, or diagnostic agent.

28. The method of claim 22, further comprising coupling the heparin or heparin-like polymer to a coupling agent represented by Formula I:
(A)ₚ(R)(D)_{q} Formula I
wherein
A is a bond or a moiety that can react with the heparin or the heparin-like polymer,
D is a bond or a moiety that can react with the biocompatible polymer,
R is a linker for A and D, and
p and q are integers from 1 to 25.

29. The method of claim 28, wherein A, for each occurrence, independently includes an amino containing group, an hydroxyl containing group, a thiol containing group, a carboxylic acid containing group, and combinations thereof.

30. The method of claim 28, wherein A, for each occurrence, includes an amino containing group.

31. The method of claim 28, wherein D, for each occurrence, includes a Michael acceptor.

32. The method of claim 28, wherein D, for each occurrence, independently includes a maleimide containing group, a vinyl sulfone, vinyl sulfoximine, isocyanate, an acrylate group, a methacrylate group, a styrene group, an acrylamide group, a methacrylamide group, acrylonitrile, a maleate group, a fumarate group, an itaconate group, a vinyl ether group, an allyl ether group, an allyl ester group, a vinyl ester group, a cinnamate group, a cyanoacrylate, a vinyl ketone, a nitro ethylene, a α,β-unsaturated aldehyde, a vinyl phosphonate, a vinyl pyridine, an azo compound, a β-keto acetylene, an acetylene ester, and combinations thereof.

33. The method of claim 28, wherein the linker R includes oxygen, sulfur, carbon, boron, nitrogen, substituted or unsubstituted alkoxy, substituted or unsubstituted alkyl, substituted or unsubstituted alkenyl, substituted or unsubstituted cycloalkyl, substituted or unsubstituted cycloalkenyl, substituted or unsubstituted aryl, substituted or unsubstituted heterocycloalkyl, substituted or unsubstituted heteroaryl, substituted or unsubstituted ether, substituted or unsubstituted amine, and a polymer.

34. The method of claim 28, wherein the coupling agent is an aminoalkyl maleimide.

35. The method of claim 22, wherein the biocompatible polymer comprises a thiol group.
